(19) United States
(12) Patent Application Publication
(10) Pub. No.: US 2022/0064831 A1
(43) Pub. Date: Mar. 3, 2022

(54) COMPUTER-AIDED PROCESS FOR CREATING A BRAIDING PROGRAM, COMPUTER PROGRAM FOR CREATING A BRAIDING PROGRAM, BRAIDING PROGRAM AND DEVICE FOR CREATING A BRAIDING PROGRAM

(71) Applicant: Admedes GmbH, Pforzheim (DE)

(72) Inventors: Florent BUDILLON, Pforzheim (DE); Kevin LEHMANN, Pforzheim (DE); Marc O. BRAEUNER, Pforzheim (DE)

(21) Appl. No.: 17/465,270

(22) Filed: Sep. 2, 2021

(30) Foreign Application Priority Data
Sep. 3, 2020 (DE) 10 2020 005 407.2

(51) Int. Cl. D04C 3/48; A61F 2/90; G05B 19/4155; G06F 9/445

(52) U.S. Cl. CPC D04C 3/48; A61F 2/90; G05B 2219/45194; G06F 9/44505; A61F 2240/001; G05B 19/4155

(57) ABSTRACT

Computer-aided process for creating a braiding program is disclosed herein. The braiding program comprises machine-readable commands, which, during execution of the braiding program, induce a braiding machine to produce a braided structure, in particular an at least partially braided implant, the process comprising: a) generating a braided structure by braiding, in particular by braid-wrapping at least one braid mandrel using a braiding material according to a specified configuration of commands; b) capturing at least one parameter of the braided structure; c) reconciling the captured parameter of the braided structure using at least one pertinent nominal parameter; provided the captured parameter does not correspond to the pertinent nominal parameter: c1-a) unbraiding at least one section of the braided structure; and c1-b) changing at least part of the configuration of commands for generating a braided structure as well as repeating the steps a) to c) based on the changed configuration of commands; provided the captured parameter corresponds to the pertinent nominal parameter: c2) creating a braiding program according to the configuration of commands, in which the captured parameter corresponds to the pertinent nominal parameter.

### CROSS-REFERENCE TO RELATED APPLICATION

The present application claims priority to German Patent Application No. DE 10 2020 005 407.2, filed Sep. 3, 2020, the disclosure of which is hereby incorporated by reference in its entirety.

### BACKGROUND OF THE DISCLOSURE

The present disclosure relates to a computer-aided process for creating a braiding program, a computer program for creating a braiding program, a computer-implemented braiding program and a device for creating a braiding program, the braiding program comprising machine-readable commands, which, during execution of the braiding program, induce a braiding machine to produce a braided structure, in particular, an at least partially braided implant.

For producing a braided structure, in particular a medical implant (such as a stent), conventionally an object is braid-wrapped using a braiding material. In doing so, the braiding material is unwound from one or a plurality of bobbins, the bobbins being passed around the object to be braid-wrapped—in most cases, along periodically crossing paths. The object to be braid-wrapped (also called braid mandrel, e.g. a substantially cylindrical body), is usually relocated relative to the bobbins and parallel to the axis of rotation of the bobbin paths. Depending on the movement of the braid mandrel and/or the bobbins, a multiplicity of braided structures having varied braid patterns and/or braiding geometries can be generated. There are known braiding machines, which allow generating random braid patterns. In doing so, different paths are connected or combined to each other using a mechanical switch, in order to guide bobbins onto different paths and thereby achieving varied braid patterns.

For producing braided structures having complex shapes and/or braid patterns, elaborate and exact synchronization of the bobbin movements and the relocation of the braid mandrel is required. The quality (e.g., material and surface) of the braid mandrel and of the braiding material must also be especially taken into consideration. This results in much work for creating an appropriate braiding program, in order to control a braiding machine in such a way that a braided structure having the desired parameters results. Moreover, frequently, many testing series are carried out and control of the braiding machine is elaborately adapted using a manual method. Such an operation is very time-consuming and costly and is hardly economically viable, particularly in the case of custom-made or small series. In addition, the quality of the braid mandrel may change between braiding operations so that adaptation of the braiding machine controls may also become necessary.

It is, therefore, an objective of the present disclosure to provide a computer-aided process for creating a braiding program. In particular, it is an objective of the present disclosure to provide a process for creating a braiding program, which allows flexible and cost-effective reasonable use.

### BRIEF DESCRIPTION OF THE DISCLOSURE

The above objective is achieved by the subjects of the independent claims. Advantageous embodiments are the subject of the subclaims.

Accordingly, in one aspect, the present disclosure is directed to a computer-aided process for creating a braiding program, the braiding program comprising machine-readable commands, which, during execution of the braiding program, induce a braiding machine to produce a braided structure (G), in particular, an at least partially braided implant, the process comprising: a) generation of a braided structure (G) by braiding, in particular by braid-wrapping at least one braid mandrel (4) using a braiding material (F) according to a specified configuration of commands; b) capturing at least one parameter of braided structure (G); c) econciling the captured parameter of braided structure (G) with at least one pertinent nominal parameter; provided the captured parameter does not correspond to the pertinent nominal parameter: c1-a) unbraiding at least one section of braided structure (G); and c1-b) changing at least part of the configuration of commands for generating a braided structure (G) as well as repeating the steps a) to c) based on the changed configuration of commands; provided the captured parameter corresponds to the pertinent nominal parameter; c2) creating a braiding program according to the configuration of commands, in which the captured parameter corresponds to the pertinent nominal parameter.

In another aspect, the present disclosure is directed to a computer program comprising commands which, during execution of the program by a device, induce the latter to execute the process for creating a braiding program as described above.

In yet another aspect, the present disclosure is directed to a device (1) for creating a braiding program, the braiding program comprising machine-readable commands, which, during execution of the braiding program, induce a braiding machine (1) to produce a braided structure (G), in particular an at least partially braided implant, the device (1) being particularly suitable for implementing the process described above and comprising: at least one bobbin (K) for guiding a braiding material (F), said at least one bobbin (K) being relocatable to move along paths, in particular of randomly specifiable paths; a relocation device (2) for relocating said at least one bobbin (K) along a path; a puller (6) for relocating braided structure (G), in particular for picking up and relocating at least one braid mandrel (4); a control unit for controlling the relocation of said at least one bobbin (K) and/or relocation of braided structure (G), in particular of said at least one braid mandrel (4), in order to generate in this manner a braided structure (G) by braiding, in particular, by braid-wrapping at least one braid mandrel (4) using the braiding material (F); and a testing means (18) for capturing at least one parameter of a braided structure (G) generated by device (I); the control unit being furthermore arranged for: a) generating a braided structure (G) by braiding, in particular by braid-wrapping at least one braid mandrel (4) using a braiding material (F) according to a specified configuration of commands; b) capturing at least one parameter of braided structure (G); c) reconciling said at least one captured parameter of braided structure (G) with at least one pertinent nominal parameter; provided the captured parameter does not correspond to the pertinent nominal parameter: c1-a) unbraiding at least one section of braided structure (G); and c1-b) changing at least part of the configuration of commands for generating a braided structure (G) as well as repeating the steps a) to c) based on the changed configuration of commands; provided the captured parameter corresponds to the pertinent nominal parameter: c2) creating a braiding program according to the configuration of commands, in which the captured parameter corresponds to the pertinent nominal parameter.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 shows an exemplary braiding machine for producing a braided structure, the braiding machine being suitable for implementing the process and the programs according to the aspects of the present disclosure;

FIG. 2 shows an exemplary arrangement of a braiding machine for producing a multiplicity of different braided structures;

FIG. 3A shows an exemplary braided structure generated by a braiding machine, on a braid mandrel;

FIG. 3B shows examples of braid bindings as braid pattern parameters of a braided structure;

FIG. 4A shows exemplary parameters of a braided structure, based on which a reconciliation with a pertinent nominal parameter can take place;

FIG. 4B shows a braided structure with exemplary parameter deviations;

FIGS. 5A-5D show exemplary braided structures, which are producible using processes, programs, and/or devices according to aspects of the present disclosure; and

FIG. 6 shows steps of a computer-aided process for creating a braiding program according to an aspect of the present disclosure.

### DETAILED DESCRIPTION

Unless defined otherwise, all technical and scientific terms used herein have the same meaning as commonly understood by one of ordinary skill in the art to which the disclosure belongs.

A first aspect of the disclosure relates to a computer-aided process for creating a braiding program, the braiding program comprising machine-readable commands, which, during execution of the braiding program, induce a braiding machine to produce a braided structure, in particular, an at least partially braided implant, the process comprising: a) generation of a braided structure by braiding, in particular, by at least partial braid-wrapping of a braid mandrel and/or at least a standing thread/standing wire using a braiding material according to a specified configuration of commands; b) capture of at least one parameter of the braided structure; c) reconciliation of the captured parameter of the braided structure with at least one pertinent nominal parameter; if the captured parameter does not correspond to the pertinent nominal parameter: c1-a) unbraiding at least one section of the braided structure; and c1-b) changing at least part of the configuration of commands for generating a braided structure as well as repeating the steps a) to c) based on the changed configuration of commands; if the captured parameter corresponds to the pertinent nominal parameter: c2) creating a braiding program according to the configuration of commands, in which the captured parameter corresponds to the pertinent nominal parameter.

This advantageously allows automated verification of the generated braided structure and automated creation and/or adaptation of the control of the braiding operation. Moreover, in this way, discarding of defective braided structures can be avoided, or at least be significantly reduced, since a braided structure with a detected parameter deviation will be corrected during the production process already.

Changing at least part of the configuration of commands is preferred dependent upon: type of the deviating parameter; extent of the deviation of the parameter; number of bobbins, type of braiding material; thickness of braiding material; surface quality of the braiding material; surface quality of a braid mandrel; and/or geometry of a braid mandrel.

One or a multiplicity of the aforementioned exemplary magnitudes and/or values must preferably be taken into consideration in changing the configuration of commands because these parameters and/or values in particular exert an at least insignificant ascertainable influence on the braided structure to be generated. Different braiding materials for example, i.e., braiding material of varied material, varied thickness (diameter), etc., require an appropriately adapted change of the configuration of commands, in order to achieve a specific adaptation of the braid pattern of the braided structure thereby created. An application of a specific tensile force (for example, value, at which the braiding material is held taut during the braiding operation) to a braiding material having a thinner diameter has a stronger effect than an application of the same tensile force to a braiding material having a greater diameter. In other words, in order to attain a specific change of the braid pattern of a braided structure, which (pattern) is particularly composed of the braid binding, the braid angle, and/or the braiding profile, with a thinner braiding material, a smaller change of a magnitude or a value (for example, the tensile force) might be sufficient, while, with a thicker braiding material, a greater change would be required. Accordingly, a similar rule applies to other magnitudes and values, which affect the braid pattern of a braided structure and which are not explicitly listed herein.

According to a particularly preferred embodiment, the change of the configuration of commands is influenced by a teachable computer-implemented algorithm. Examples thereof are machine-learning and artificial intelligence, in particular monitored learning, as well as programs, which are capable of (semi)autonomous behavior and evaluating specific actions.

Particularly, in the case of large amounts of data, the human ability to absorb these data, to interpret them, and to make complex decisions based on them is exceeded. Artificial intelligence and/or machine learning forms the basis of learning computer systems and is capable of implementing complex decision-making processes. Above all, in the case of a large volume of possible combinations and/or permutations of parameters, values and/or variables, such systems and algorithms can be used for finding an optimal, or at least advantageous and/or faster solution. This also applies to a preferred embodiment of the present disclosure, since, inter alia, a large number of parameters exists, which influence the finished braided structure. Likewise, an abundance of possible changes and combinations of one or a plurality of these parameters exists.

In comparison to an algorithm that is based on artificial intelligence/machine learning, an iterative and/or incidental change of one or a plurality of parameters, for example, frequently brings about a satisfactory solution only tediously or not at all. By using artificial intelligence/machine learning, it is advantageously possible to arrive at a solution faster and/or obtain a superior solution.

Moreover, in producing a braided structure, it is difficult or impossible to simulate the effects of parameter changes by computer assistance so that effects of parameter changes on the braiding result can frequently only be ascertained by an appropriately implemented braiding operation. This can, however, cause a substantial amount of extra work. Using artificial intelligence/machine learning, the number of braiding operations required for finding an optimal, or at least advantageous, solution can frequently be reduced.

The teachable computer-implemented algorithm can, for example, change the configuration of commands based on: a specified configuration of commands; a previously captured parameter deviation; an implemented configuration change; and/or a detected effect of a specific configuration change.

Preferably, the teachable, computer-implemented algorithm is arranged for accessing information and to take it into consideration in automatically changing the configuration of commands. Particularly preferred in this arrangement is an algorithm that has access to specified configurations of commands for generating a braided structure and/or to one or a plurality of previously created braiding programs. Alternatively, and/or additionally, it is advantageous that the algorithm be capable of accessing information concerning one or a plurality of specified and/or captured parameter deviations and/or furthermore preferred, one or a plurality of specified and/or implemented configuration changes. Advantageously, the algorithm can achieve a solution and/or find an enhanced/preferred solution faster in this manner.

Even more suitably, the configuration of commands for generating the braided structure comprises one or a plurality of the following parameters: relocation of at least one bobbin; relocation of the braided structure; relocation of a braid mandrel; tension of the braiding material, position, and/or relocation of at least one braid ring and/or one braid eye.

As described above, a braiding operation is particularly defined and/or controlled by the specification of one or a plurality of listed exemplary parameters. The configuration of commands for generating the braided structure preferably comprises a chronologically and/or spatially synchronized sequence of commands, which control at least one or a plurality of the indicated parameters.

Advantageously, one parameter of the braided structure comprises: position of at least one crossover of the braiding material (for example, in relation to one or a plurality of other crossovers and/or at least one feature of a braid mandrel and/or one or a plurality of random other reference points, which are not part of the braided structure); diameter of the braiding material; length of a rhombus; width of a rhombus; length of a rhombus segment; and/or braid angle of a rhombus. Additional exemplary parameters are rhombus area, rhombus density, and braided structure diameter.

A reconciliation of a position of a crossover of the braided structure in relation to one or a plurality of (profile) features of a braid mandrel is particularly advantageous with a profiled braid mandrel (e.g., a braid mandrel having one or a plurality of changes of the mandrel diameter), because, in this way, particular nodes/crossovers in the area of a change of the mandrel profile can be generated or placed exactly or with as small a deviation as possible.

The capture of a parameter of the braided structure preferably takes place in at least one specific section of the braided structure, preferably in relation to a braid mandrel, in particular in an aerial section, in a dropping section, and/or in a fastening section of the braided structure.

Preferably, unbraiding comprises a substantially reverse relocation of at least one bobbin and/or said at least one braid mandrel. Particularly preferred, said at least one bobbin is enhanced in such a way so as to allow both generating a braided structure and also unbraiding at least part of a braided structure, for example by returning the braiding material into a material storage device. A particularly preferred enhancement of such a bobbin is described in patent specification DE 10 2011 118 108 B3, the contents of which are hereby incorporated by reference.

Thus, advantageously unbraiding is possible because no additional means (beyond an appropriately enhanced bobbin) needs to be provided for unbraiding.

Furthermore preferred, unbraiding takes place at least up to the section of the braided structure, which comprises the parameter that deviates from the pertinent nominal parameter.

A second aspect of the disclosure relates to a computer program comprising commands, which, during execution of the program by a device, induce the latter to execute the process for creating a braiding program according to the first aspect of the disclosure.

A third aspect of the disclosure relates to a computer-implemented braiding program comprising commands, which, during execution of the program, induce a braiding machine to produce a braided structure, in particular, an at least partially braided implant, the braiding program having been created by the process according to the first aspect of the disclosure.

A fourth aspect of the disclosure relates to a device for creating a braiding program, the braiding program comprising machine-readable commands, which, during execution of the braiding program, induce a braiding machine to produce a braided structure, in particular an at least partially braided implant, the device for executing the process according to the first aspect of the disclosure being suitable and comprising: at least one bobbin for guiding a braiding material, said at least one bobbin being relocatable to move along paths, in particular, of randomly specifiable paths; a relocation device for relocating said at least one bobbin along a path; a puller for relocating the braided structure, in particular, for picking up and relocating at least one braid mandrel; a control unit for controlling the relocation of said at least one bobbin and/or relocation of said at least one braid mandrel, in order to generate in this manner a braided structure by braiding, in particular, by at least partial braid-wrapping of said at least one braid mandrel using the braiding material; and a testing means for capturing at least one parameter of a braided structure generated by the device; the control unit being furthermore arranged for: a) generating a braided structure by braiding, in particular, by at least partial braid-wrapping at least one braid mandrel using a braiding material according to a specified configuration of commands; b) capturing at least one parameter of the braided structure; c) reconciling said at least one captured parameter of the braided structure with at least one pertinent nominal parameter; if the captured parameter does not correspond to the pertinent nominal parameter: c1-a) unbraiding at least one section of the braided structure; and c1-b) changing at least part of the configuration of commands for generating a braided structure as well as repeating steps a) to c) based on the changed configuration of commands; if the captured parameter corresponds to the pertinent nominal parameter: c2) creating a braiding program according to the configuration of commands, in which the captured parameter corresponds to the pertinent nominal parameter.

A relocation of the braided structure by a puller is preferably carried out directly, for example, by spatial shifting of the generated braided structure and/or indirectly, for example, by spatial shifting a braid mandrel, around which the braided structure was generated.

Hereinafter, individual embodiments for achieving the objective are described as examples based on the figures. Therein, part of the individually described embodiments have features which are not absolutely required for executing the claimed object, but which provide desired characteristics in certain applications. In this way, embodiments shall even be considered as having been disclosed as corresponding to the described technical teaching if they do not have all the features of the embodiments described hereinafter. Furthermore, in order to avoid unnecessary repetitions, certain features will only be mentioned with reference to individual embodiments among the embodiments described hereinafter. It should be noted that the individual embodiments should, therefore, not only be viewed per se but also in a synoptic overview. Based on this synoptic overview, the person skilled in the arts will recognize that individual embodiments can also be modified by inclusion of individual or multiple features of other embodiments. It should be noted that a systematic combination of the individual embodiments, having individual or multiple features, which are being described in relation to other embodiments, may be desirable and useful and should, therefore, be taken into consideration and should also be considered as being comprised by the description.

FIG. 1 shows an exemplary and preferred device 1 according to an embodiment of an aspect of the present disclosure. The shown device 1 has a relocation device 2 for relocating one or a plurality (e.g., 2) of bobbins K along a specific path. Further preferred, device 1 has a rotary disc with additional winged wheels, on each of which at least one bobbin K is guided so that complex and individual paths of the bobbins K are made possible.

Optionally, device 1 can have one or a plurality of braid rings 8 and/or a braid eye 10—as shown in FIG. 1. Such a braid ring 8 is preferably arranged between a bobbin K and a braid mandrel 4, in order to turn the braiding material F exiting from bobbin K toward braid mandrel 4. Preferably, a braid ring 8 and/or a braid eye 10 is relocatable, in order to change an angle of the braiding material F during the drop on braid mandrel 4. Braiding material F will preferably be pulled off spindles and/or spools of the bobbins K. It is also conceivable for braiding material F from a material storage device (not shown) to be fastened to bobbin K and/or guided and/or tensioned by means of a connecting element, as for example shown in patent specification DE 10 2014 016 381 B4, which is incorporated by reference. For control and/or maintenance of the tension of braiding material F, device 1 can preferably have weight and/or spring systems. Advantageously, the tension of braiding material F can be individually controlled and/or dynamically changed or adapted during the braiding process.

Device 1 is preferably suitable for the production of at least partially braided body implants or stents. They protect channels of living bodies such as, for example, blood vessels, esophagus, urethra, or kidney ducts by introducing the stent and expanding the stent inside the body channel. In this way, a collapse or blockage of the body channels concerned can be prevented. Moreover, a stent will, for example, be used for intracerebral vascular bulges, so-called aneurysms, which are the most frequent cause of non-traumatic subarachnoid hemorrhaging. Their incidence in the total population is around 1%, according to autopsy studies, even as high as 9%. Pathomorphologically, intracerebral aneurysms are, as a rule, true sacural aneurysms, which are mostly located in vascular ramifications (see for example Schumacher M., “Diagnostic work-up in cerebral aneurysms” in Nakstadt PHj (ed): “cerebral aneurysms”, pp 13-24, Bologna: Centauro (2000)).

Moreover, such body implants or stents can be used as drug carriers to make local therapy within the body channel possible.

As braiding material F, for such body implants, nitinol will preferably be used, which will have preferably undergone a thermal treatment in a saline bath, in order to impress specified shape memory characteristics into the nitinol. Nitinol is a nickel-titanium alloy having shape memory characteristics and an orderly-cubic crystal structure, which differs from that of titanium and nickel. It consists primarily of nickel (approx. 55%) and an additional large proportion of titanium. The alloy is corrosion-resistant, high strength and super elastic. Due to the excellent deformability and the good corrosion resistance, surgical tools, endoscopes, or implants such as, for example, stents are a typical application. An alloy having a high transformation temperature of 80° C., for example, is also referred to as memory metal or shape memory metal. A multiplicity of other materials such as stainless steel, for example can, however, also be used.

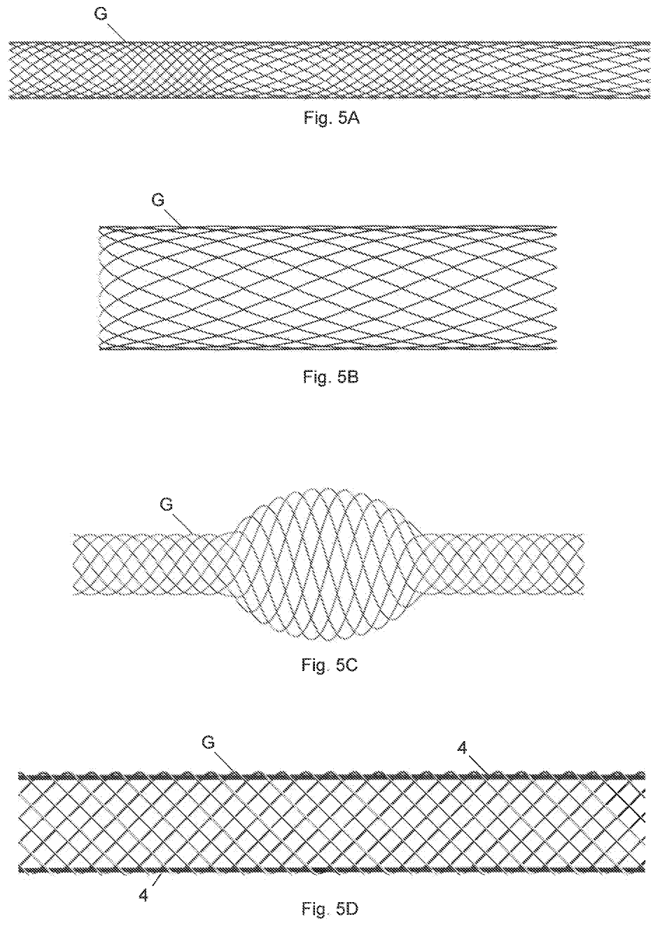

Preferably, device 1 has a puller 6 for relocating the braided structure, in particular for picking up and relocating a braid mandrel 4. Puller 6 is preferably suitable for relocating the braided structure and/or a braid mandrel 4 in a linear direction relative to the bobbins K and/or parallel to the axis of rotation of the bobbin paths. By such a relative relocation, braided structure G is generated, for example along or on the surface of braid mandrel body 4. Relocation of the one or the plurality of bobbins K and/or of the braided structure G or a braid mandrel 4 takes place according to a configuration of commands, which are provided by a control unit S and which appropriately control and regulate relocation device 2 and/or puller 6. In this manner, a specific braided structure G is generated by braiding, in particular, by at least partial braid-wrapping of braid mandrel 4 using braiding material F. In an analogous manner, device 1 is suitable for generating a flat braided structure, in particular, by braiding material F at or around or along at least part of at least one braid mandrel 4 and/or of one or a plurality of standing threads/wires and relocating the braided structure or said at least one braid mandrel 4 (see, for example, FIG. 5D.)

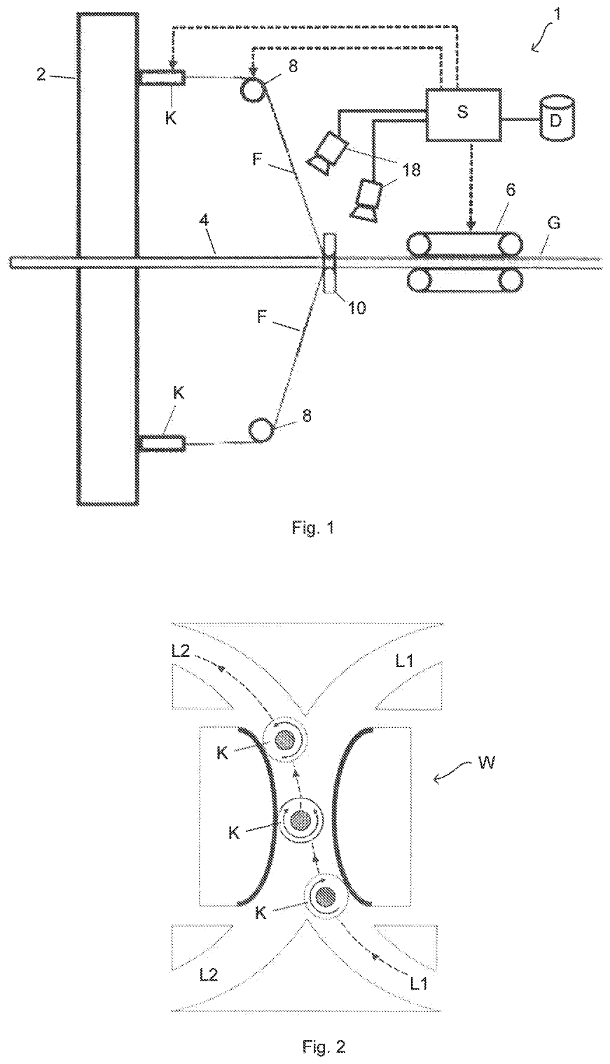

Further preferred, device 1 allows generating random braid patterns. In doing so, for example different paths of bobbins K are connected to each other or combined by means of a mechanical switch W to guide bobbins K on different paths and thereby to achieve varied braid patterns. One such operation is shown in FIG. 2.

Moreover, device 1, according to the shown especially preferred embodiment, has at least one testing means 18 for checking braided structure G. In particular, the one or the plurality of testing means 18 are suitable for capturing one or a plurality of parameters of braided structure G and/or to reconcile with a pertinent nominal parameter in each case. In this manner, device 1 can check whether any adaptation or change of the configuration of commands, according to which braided structure G is generated, should be carried out. Such a testing means 18 can, for example have a camera, a microscope, a laser scanner, a measuring head, a computer tomograph instrument, and/or other devices suitable for capturing one or a plurality of parameters of the braided structure. In particular, several identical and/or varied testing means 18 can be provided. The testing operation can be carried out substantially continuously, at a constant and/or variable rate.

More preferably, device 1 has a control unit S. Control unit S is advantageously signal-linked to the one or the plurality of testing means 18 and arranged so as to receive information about one or a plurality of the captured parameters of the braided structure G (e.g., via line(s) and/or wirelessly). Advantageously, control unit S has access to a data base D, data base D, for example, comprising configurations of commands for generating a braided structure, one or a plurality of braiding programs for controlling a braiding machine, and/or information concerning known or specified parameters or parameter deviations. In particular, control unit S is arranged in such a way that it is arranged for reconciling one or a plurality of captured parameters of braided structure G using at least one specifiable or specified nominal parameter. Further preferred, control unit S is arranged for controlling or regulating the relocation of said at least one bobbin K and/or of the braided structure G and of said at least one braid mandrel 4. Further preferred, control unit S can relocate one or a plurality of relocatable braid rings 8 and/or control or regulate features of a braid eye 10 (such as, for instance, inside diameter, inlet radius, material, surface quality, tribological characteristics, position and/or orientation).

Moreover, according to a particularly preferred embodiment, control unit 8 of device 1 is suitable for at least partially changing a configuration of commands, based on which a braided structure G is generated by braiding, in particular by at least partially braid-wrapping a braid mandrel 4, and further preferred, to control device 1 according to the changed configuration of commands.

FIG. 2 shows an exemplary process for the complex relocation of at least one bobbin K, for which purpose a particularly preferred device 1 is arranged. In this exemplary process, a transfer or a transfer operation of a guide element of a bobbin K from path L1 of a right-hand winged wheel (not illustrated) to path L2 of a left-hand winged wheel (not illustrated) takes place. After entry of the guide element into switch W, the guide element is transferred to the left-hand winged wheel. This can, for example, take place by appropriately controlled electromagnets and/or mechanical actuators in the switch area. In this way, the guide element and consequently bobbin K is transferred from a path L1 to another path L2. Relocation device 2 of device 1 according to FIG. 1 preferably has one or a plurality of such or of alternative arrangements for the individual and adaptable relocation of one or a plurality of bobbins K along a complex path. A particularly preferred embodiment of a device or a process for relocating at least one bobbin K is described in patent specification DE 10 2016 013 486 B3, the contents of which are hereby incorporated by reference.

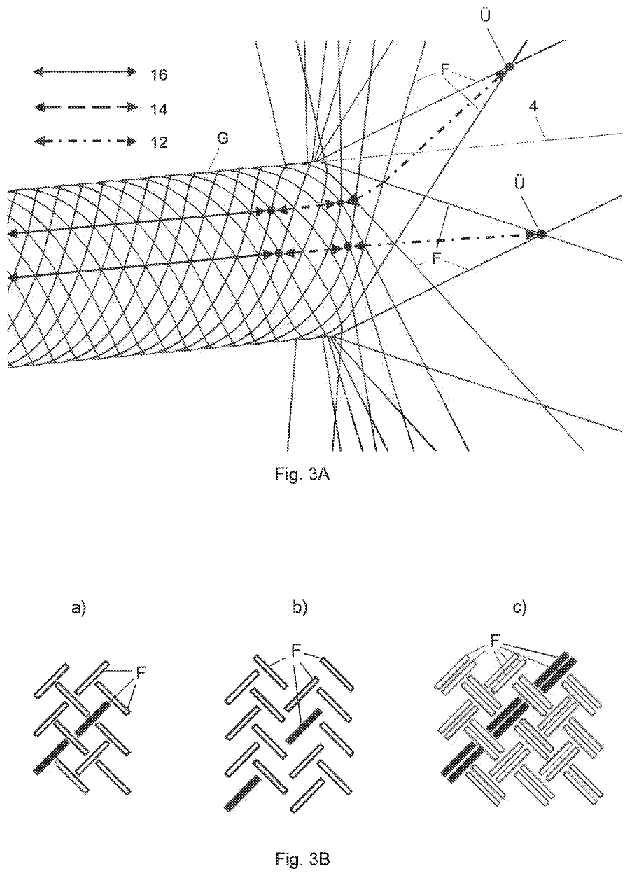

With reference to FIG. 3A, the one or the plurality of testing means 18 of device 1 are preferably arranged in such a way that one or a plurality of sections of the braided structure can be checked. Preferably, at least that section of braided structure G can be checked, in which braiding material F is substantially fastened or which is located on a braid mandrel 4 and/or in which braiding material F is substantially fastened on the braid mandrel (fastening section 16.) Alternatively, and/or additionally, the one or the plurality of testing means 18 are arranged in such a way that it can at least check that section of braided structure G, in which braiding material F abuts the braided structure or drops at/on at least one standing thread/standing wire or on braid mandrel 4 (dropping section 14.) In dropping section 14 of braided structure G, the braid pattern can further change by a further relocation of the bobbins K and/or braid mandrel 4. Further preferred, at least one testing means 18 is arranged in such a way that alternatively, and/or additionally to fastening section 16 and/or dropping section 14, the aerial section 12 of braided structure G is checked. The aerial section 12 of the braided structure is situated ahead of the dropping section 14 and begins, for example, at the point, at which a crossover of braiding material F forms in the air.

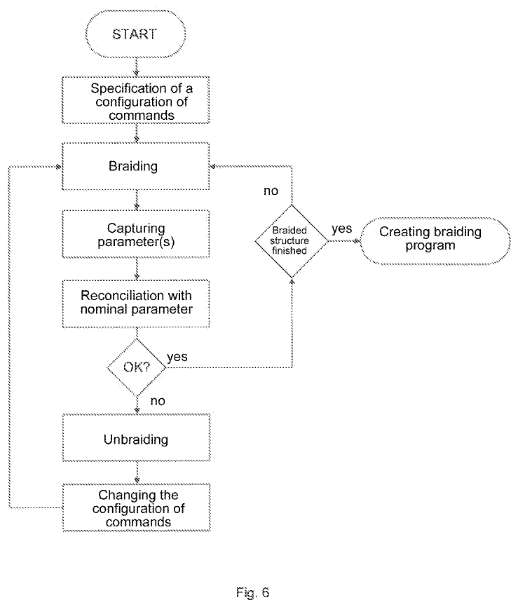

According to an additional particularly preferred embodiment, at least one testing means 18 and the control unit S are arranged in such a way that deviating parameters of braided structure G will be recognized as early as possible so that only as small an area as possible of braided structure G needs to be unbraided for correcting the deviating parameter (as will be described in greater detail hereinafter with reference to FIG. 6).

Furthermore, it is advantageous if control unit S is arranged to predict a developing parameter deviation of braided structure G and to potentially prevent it by adapting the relocation of bobbin K and/or the braided structure and/or braid mandril 4 (real-time correction). Alternatively, and/or additionally, the braiding operation can be stopped early, for instance, even before the drop of the section of braided structure G on braid mandrel 4, which has the deviating parameter or will have it. In this way, a reduction of the section of braided structure G to be unbraided and as a result, a decrease of the correction work, can be achieved. To achieve this, it is particularly advantageous to provide one or a plurality of testing means 18. Preferably, the one or the plurality of testing means 18 check varied and/or overlapping sections of braided structure G or of braiding material F, as described with reference to FIG. 3A. Moreover, preferably, different testing means 18 can be provided in order to capture different parameters of braided structure G or of braiding material F in each case. Also, at least one testing means 18 can be provided, in order to detect a crossover or a node U of braiding material F (compare FIG. 3A) prior to dropping braiding material F on braid mandrel 4.

Advantageously, control unit S of device 1 is further arranged to carry out a reconciliation between a parameter of the braided structure captured by a testing means 18 and a pertinent, specified, or specifiable (nominal) parameter. The nominal parameter is for example specified or defined by the specified or desired braid pattern of the braided structure G to be produced. The braid pattern is particularly defined by one or a plurality of the following braid pattern parameters: number and/or diameter of the braiding material F), braid binding (e.g. 1:1-1, 1:2-1, 1:1-2, etc.), braided structure diameter 32, braided structure profile, length of the braided structure or certain sections of the braided structure, braid angle (angle between braiding materials F, or braiding material F and longitudinal axis of braid mandrel 4), braiding density (number of geometric shapes (e.g., rhombuses) formed by the braiding material in a defined subsection or section of the braided structure).

In FIG. 3B, exemplary braid bindings are illustrated, which particularly characterize a braid pattern of a braided structure G (braid pattern parameter). In order to allow distinguishing and classifying different braided structures, an appropriate notation system is advantageous. In FIG. 3B, exemplary notations of braid bindings—in particular, those known in Germany—are illustrated. Preferably, the name of the braid binding has two components: the first part, for instance, describes the crossover shape, the second part describes the number of braiding threads used. The braid bindings shown correspond to preferred or common braid patterns: a) single plait single thread—1:1-1; b) double plait single thread—1:2-1; c) single plait double thread—1:1-2. A braided structure G may have a uniform braid binding or varied braid bindings in different sections. The braid binding is selectable according to the characteristics to be achieved or desired, such as for example shape, permeability and/or flexibility, of the braided structure G or of sections of braided structure G. Depending on the braid binding, varied configurations of commands for producing a braided structure are provided or used. The braid bindings illustrated in FIG. 3B show some braid bindings for illustration purposes, the device and the process according to the present disclosure being also suitable for generating additional braid bindings not shown, in particular including braid bindings of other notations.

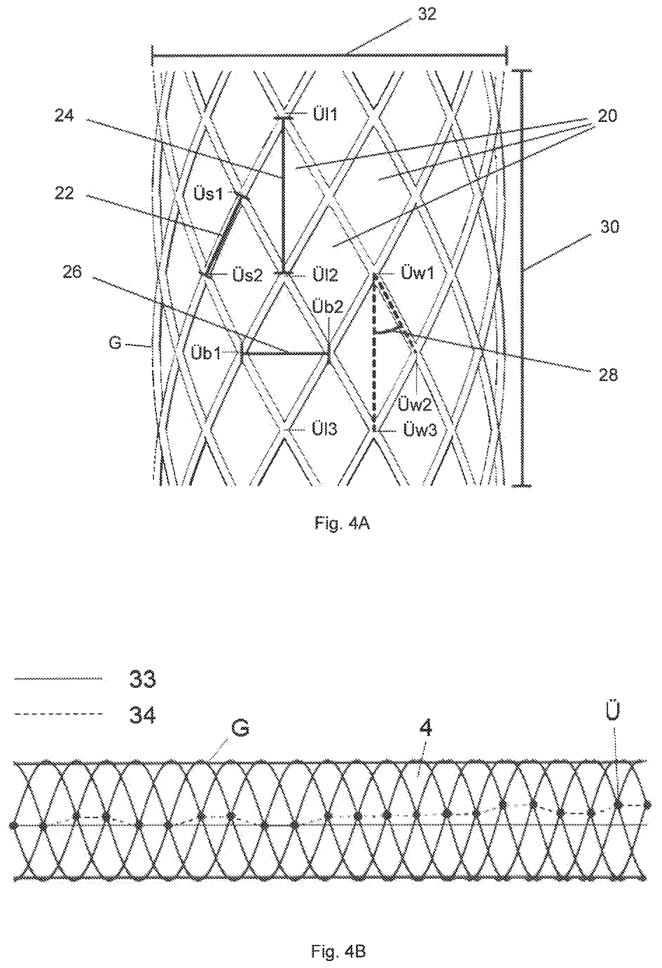

Hereinafter, some of the parameters and/or braid pattern parameters particularly preferred and illustrated as examples in FIG. 4A, are exemplified:

The indicated exemplary parameters denote preferred geometric or structural features and/or characteristics of a braided structure G. A crossover or a node U of braiding material F is, for example, formed by a crossover of two braiding materials or braiding thread F of varied bobbins. A plurality of crossovers or nodes U (e.g., four) form a geometric shape, for example a rhombus 20.

A rhombus segment or a rhombus web 22 preferably corresponds to a section of a braiding thread F, which connects two adjacent crossovers or nodes Ü of the same braiding thread F. In FIG. 4A, this is exemplified by a web 22 between nodes or crossovers Üs1 and Üs2, Üs2 and Üb1 or Üs1 and Ül2.

The length 24 of a rhombus 20 preferably corresponds to the separation or the distance between two crossovers or nodes U, in each case of two varied braiding materials or braiding threads F, which are essentially arranged opposite each other in the axial direction of mandrel 4 or of braided structure G. These crossovers or nodes U are preferably connected by two rhombus segments 22 of varied braiding threads F. In FIG. 4A, this is exemplarily the separation between nodes Ül1 and Ül2, Ül2 and Ül3 and/or Üw1 and Üw3.

Similar to length 24 of a rhombus 20, width 26 of a rhombus 20 preferably corresponds to the separation or distance between two crossovers or nodes U, each of two varied braiding threads F, which are, however, substantially arranged opposite each other in the radial direction of mandrel 4 or of braided structure G. In FIG. 4A, this is exemplified by the separation between nodes Üb1 and Üb2, Üb2 and Üw2 and Ül3 and Üw3.

Braid angle 28 of a rhombus 20 preferably corresponds to the angle of a rhombus segment 22 relative to the axial or the radial direction of braided structure G or braid mandrel 4. Alternatively, a braid angle 28 may describe the angle, which is formed between two rhombus segments 22. In FIG. 4A, this is exemplified by the angle between segment 22, which is formed by the two nodes Üw1 and Üw2 and the axial direction of braid mandrel 4 or braided structure G. Another example is the angle between segment 22, which is formed by the two nodes Üw1 and Üw2 and segment 22, which is formed by the two nodes Üw2 and Üw3. However, the rhombus segments 22, which define braid angle 28, need not necessarily have a joint node U. A braid angle 28 can, for example, also be defined between segments 22 of a rhombus 20 situated opposite each other or between segments 22 of different rhombuses 20.

The rhombus density preferably corresponds to the number of rhombuses over a defined length, width, and/or surface of at least one subsection of braided structure G. In FIG. 4A, this is exemplified by the number of rhombuses 20 adjoining each other in the axial direction over the length of the illustrated subsection 30 of braided structure G. Preferably, the corresponding subsection 30 is selected in such a way that it begins, for instance, at a first node U of a rhombus and extends along a specified direction (e.g., an axial direction of braid mandrel 4 or of braided structure G) over a specified length or distance. The value of the rhombus density is, for example, the number of established nodes or crossovers U (e.g., in the axial direction, the foremost node of a rhombus ÜL1) in the established subsection 30 of the braided structure. The same applies to an area or a width of a subsection 30 of braided structure G.

The parameters shown and/or listed correspond only to a selection of possible suitable parameters of braided structure G. Rhombus 20 is a generated shape that occurs frequently among braided structures, in particular at least partially braided implants, and is only mentioned as an example in the present disclosure. In particular, depending on the number of braiding threads F or bobbins K and their relocation, other shapes can also be created, making possible additional and/or alternative parameters for capturing and reconciling.

Reconciling the parameter will, for example, involve checking whether one or a plurality of captured parameters in each case correspond to a pertinent specified or specifiable nominal parameter. Particularly preferred is a substantially exact agreement of the captured parameter with the pertinent nominal parameter. There may, however, exist tolerance ranges, in which a deviation of the captured parameter from the nominal parameter can fluctuate, without the result of a reconciliation being negative. Depending on the type of parameter, measuring accuracy of the testing means 18 and/or of specified or desired fabricating accuracy, these tolerance ranges may vary (for example, in the range of less than 20%, preferably less than 5%, more preferably less than 1%). If the deviation of a parameter is within the defined tolerance range, a positive result of the reconciliation exists.

Device 1 described in connection with FIG. 1 is moreover advantageously suitable for unravelling and/or unbraiding at least a section of the generated braided structure G. For unbraiding to be feasible, control unit S is preferably arranged to appropriately control or regulate bobbin K, braided structure G, braid mandrel 4, switches W, braid rings 8 and/or braid eye 10. In doing so, for example one or a plurality of bobbins K and/or braided structure G or braid mandrel 4 are relocated along the corresponding path in the opposite direction in such a way that ordered unbraiding of at least one section of braided structure G is achieved. Should a negative result of a reconciliation of a captured parameter from a nominal parameter exist, i.e., a deviation of the parameter outside the defined tolerance range, the braiding operation can thus be stopped and preferably be unbraided at least up to the section of braided structure G, which contains the deviating parameter.

Control unit S of device 1 is advantageously arranged to change or adapt the configuration of commands, according to which braiding, in particular the braid-wrapping of braid mandrel 4, is carried out. One or a plurality of parameters of at least part of the configuration of commands can thus be changed by control unit S in such a way that any deviation of a captured parameter is prevented or a captured parameter is caused to approach a pertinent nominal parameter in such a way that it is preferably situated within a potential tolerance range. The changeable parameters of the configuration of commands comprise, for instance, those parameters, which are responsible for the relocation of one or a plurality of bobbins K, the relocation of braided structure G or of braid mandrel 4, and/or for controlling the tension of braiding material F. The relocation of a bobbin K comprises in particular a spatial path and/or the speed, at which a bobbin K is moved along a path. Likewise, for instance, the reconciliation or the timing of the relocation of two or a plurality of bobbins K can be changed, as well as a relocation of one or a plurality of braid rings 8 or control of a braid eye 10 and/or a tension of braiding material F.

According to a preferred embodiment, the change of the configuration of commands (at least partially) is carried out automatically. For this purpose, simple iterative methods or optimization algorithms such as the Gauß-Newton method and/or gradient methods are particularly suitable. However, such an approach frequently results in a large number of tests to be evaluated, with every failure another unbraiding becoming necessary. According to a particularly preferred embodiment of the disclosure, the change of the configuration of commands is, therefore, influenced or determined by a teachable computer-implemented algorithm. By using artificial intelligence/machine learning, it is advantageously possible to achieve a solution faster and/or to attain a superior solution. This allows accelerating the discovery of a suitable change, for instance, compared to the mentioned optimization methods and randomized or arbitrary parameter changes. One such algorithm is preferably capable of drawing on experience, for example, on previously detected parameter deviations, in order to correct implemented measures and the result of these measures. Information from the device of known specified configurations of commands can also be included for finding a suitable change. In this way, accelerated correction of the deviation by targeted and promising changes of the configuration of commands controlling the braid-wrapping can be achieved.

Advantageously, changing at least part of the configuration of commands depends on the type of deviating parameter, the extent of the deviation of the parameter, the type of braiding material F, the thickness of braiding material F, the surface quality of braiding material F, the surface quality of braid mandrel 4 and/or the geometry of braid mandrel 4 for example. These and similar characteristics and parameters particularly influence the braiding result.

For instance, in case the parameter of rhombus length 24 captured by one or a plurality of testing means 18 is too small: in this case, an increase in the relocation speed of braided structure G or braid mandrel 4 (e.g., by appropriate control or regulation of puller 6) can, for example, be used for correction. In case of an accidental change of the configuration of commands, both the parameter or the parameters of the configuration of commands to be changed and the extent of this parameter change must be determined by an involved trial-and-error process. As a rule, this is time-consuming and costly. Using the process according to the described particularly preferred embodiment, it is, for example, possible to draw on experience and to implement a parameter change that presents as great a chance of success as possible. Advantageously, alternatively, and/or additionally, a known configuration of commands, which comprises generating a braided structure with elongating rhombuses, can be used as a basis and as supporting information in searching for a suitable parameter change. In this way, the time and/or the number of the attempts necessary for achieving the desired or specified braid pattern of braided structure G can be reduced.

Likewise, for example by a continuous change of the surface quality of braid mandrel 4 by wear, an appropriate adaptation of the braiding operation may be advantageous. The control unit of device 1 can draw on configuration changes implemented in the past and compensating the wear and can achieve a suitable adaptation of the configuration of commands faster in this manner.

In a further exemplary application, a deviation of a braid angle 28 of braided structure G is captured by one or a plurality of testing means 18 (e.g., one or a plurality of cameras). A corresponding advantageous change of the configuration of commands can, for example, comprise a change of the relocation of one or a plurality of bobbins K with simultaneous acceleration or deceleration of the relocation of braided structure G or braid mandrel 4. In arriving at a decision regarding the parameter to be changed or the parameters to be changed and/or in arriving at a decision regarding the scope and type of the change, control unit S can preferably access braiding programs, configuration of commands, and/or previously implemented or specified configuration changes stored in a data base D. In this way, control unit S can, for example, access information regarding bobbin relocation and/or braided structure or braid mandrel relocation of a braiding program for braiding a braided structure G having a continuously changing braid angle and adapt the specified or current configuration of commands based therein.

FIG. 4B shows a braided structure G with exemplary parameter deviations. The illustrated braided structure G has nodes/crossovers U, at least their position (clarified by connecting line 34) deviating in relation to braid mandrel 4 or its longitudinal axis 33. The pertinent, specified, or specifiable positions (nominal parameters) of the nodes or crossovers U of braided structure G are located on or along longitudinal axis 33 of braid mandrel 4 in this example. The shown exemplary deviations of the “straightness of the braided structure” are correctable by changing the configuration of commands, in particular, by changing the parameters for controlling or regulating one or a plurality of braid rings 8 and/or braid eyes 10.

In each case, braid-wrapping is resumed or started using the last changed configuration of commands and is continued until an additional parameter deviation is detected or braided structure G has been completed. Should a parameter deviation be detected, a corresponding change of the configuration of commands is implemented and—after unbraiding the affected section—the generation of braided structure G by braiding, in particular, by at least partial braid-wrapping of said at least one braid mandrel 4, according to the changed configuration of commands is continued. If no (additional) parameter deviation is detected and the braided structure has been completed, a braiding program is created, which comprises the configuration of commands, in which all captured parameters correspond to the pertinent nominal parameters. The created braiding program comprises commands, which, during execution of the program, induce a braiding machine to produce a braided structure G with the desired characteristics by braiding, in particular, by at least partial braid-wrapping of at least one braid mandrel 4 using a braiding material F according to the changed configuration of commands.

The FIGS. 5A-5D show examples of braided structures G, which are producible using the processes, programs, and/or devices of the present invention according to corresponding configurations of commands. FIG. 5A shows, as an example, an open braided structure (endless braided structure), which has a substantially constant diameter and subsections with varied braid patterns. The braided structure has both close-mesh and wide-mesh subsections as well as a transition area between these subsections. The subsections differ for example by braid angle, rhombus length, and/or rhombus segment length. A stent, which has a braid pattern as shown in FIG. 5A, is particularly suitable for the treatment of or use in aneurysms, a narrow-mesh subsection being positioned at the location of the aneurysm. FIG. 5B shows a braided structure with loops at one end of the braided structure. In producing such a braided structure, it is particularly important to watch for the earliest possible setup of the desired rhombus geometry. In most cases, each of the shapes of the first rhombuses deviates from those of the following rhombuses. In the case of medical implants, such as for example stents, such or similar loops generate a restoring force at one end so that the stent is expanded at this end. FIG. 5C shows an additional exemplary braided structure, which was created on a profiled braid mandrel. In the case of medical implants, it may be advantageous if the geometry/shape of the braided structure is adapted to the shape of the vessel into which it is to be introduced. In particular in the case of a profiled braid mandrel 4 (e.g., braid mandrel 4 with one or a plurality of changes of the mandrel diameter), it is advantageous if the specified or specifiable position (nominal parameter) of a crossover U of braided structure G is capturable and reconcilable in relation to one or a plurality of (profile) features of braid mandrel 4. Such an operation is especially advantageous for generating and/or placing nodes/crossovers U in the area of a change in the mandrel profile exactly or with as small a deviation as possible.

Moreover, an unwanted deviation of the generated braided structure G by accumulating or totaling parameter deviations situated within an established tolerance may develop. By reconciling the position of one or a plurality of crossovers in relation to one or a plurality of reference points, which are not part of the braided structure, deviations of the braided structure are advantageously detectable and reducible or remediable by an appropriate correction, in particular reducible to below a specified tolerance. FIG. 5D shows an exemplary flat braided structure G (gimp), which is producible using the process and devices of the present application. Preferably, in the case of flat braided structures, braiding material F crosses over and turns around by the edges of flat braided structure G. Advantageously, at the edges of braided structure G, braiding material F is applied at least partially around or to a braid mandrel 4, more preferably, a braid mandrel 4 being provided in each case by one edge of braided structure G. Alternatively, and/or additionally, a flat braided structure may have one or a plurality of standing threads/wires, in particular, by its edges. A standing thread or a standing wire may be identical to braiding material F of the braided structure or have other characteristics (e.g., material, thickness, surface quality, etc.). A flat braided structure may have sections with varied geometries and/or braid patterns. Preferred is the ratio between one spatial extension in a first direction (thickness—perpendicular to the image plane) and in a second direction (width—top-bottom in the image plane) of a flat braided structure in the range of approximately 1:1.5 to approximately 1:100, more preferred in the range from approximately 1:3 to approximately 1:50, more preferred in the range from approximately 1:5 to approximately 1:20, the first direction (thickness) and the second direction (width) being substantially orthogonal to one another and to the longitudinal axis of braid mandrel 4. The thickness of a flat braided structure is particularly defined by the diameter of braiding material F, the braid pattern, and/or the geometric characteristics of one or a plurality of potential braid mandrels or standing threads/standing wires. The width of the flat braided structure is, for example, defined by the separation of potential braid mandrels or standing threads/standing wires, which (separation) can, for example, in turn be defined by the relocation of the braided structure or the variation of the wire tension and set as desired. The length of a flat braided structure (left-right in the image plane) can be arbitrarily determined and arbitrarily enlarged, in particular by a continuously running braiding operation (endless braided structure).

Additional braided structures (not illustrated) can, for example, comprise twists, branches, flat-braided areas, and round braided structures. Braided structures with combinations of the shown and mentioned shapes/characteristics are also producible using the process, programs, and/or devices of the present disclosure. In particular, when changing the diameter or the shape of the braid mandrel, multiple iterations of change or adaptation operations of the configuration of commands may be necessary for obtaining a braided structure having the desired or specified characteristics. Using the present disclosure, the work involved for this purpose can be reduced.

FIG. 6 illustrates steps of a preferred embodiment of the computer-aided process for creating a braiding program according to one aspect of the disclosure. The process is particularly suitable for being implemented by a device 1 according to an embodiment of the disclosure.

In a first step, a configuration of commands is made available to a control unit S, for example by a storage medium. The configuration of commands comprises commands causing, subject to the control of a control unit S, a relocation of one or a plurality of bobbins K by a relocation device 2 (e.g., a turntable and/or a winged wheel) of device 1. Preferably, the configuration of commands also comprises commands, which (configuration) controls a puller 6, for example, in order to relocate the braided structure, in particular, by relocating a braid mandrel 4 coupled with it. By execution of the commands by control unit S, in a second step, a braided structure G is generated by braiding, for example, by braid-wrapping a braid mandrel 4 using braiding material F provided by one or a plurality of bobbins K.

Preferably, during the braiding operation, in a third step, one or a plurality of parameters of the developing braided structure G are captured by control unit S and—in a fourth step—reconciled with at least one corresponding or pertinent nominal parameter. In return, control unit S receives from one or a plurality of coupled testing means 18, for example, information concerning said at least one captured parameter of braided structure G. Such a testing means 18 is, for example, a camera, preferably a high-resolution high-speed camera, which is suitable for capturing one or a plurality of parameters of braided structure G and for transmitting them to control unit S. Such a parameter is, for example, a length or width of a rhombus 20 formed by crossovers U of braiding material F. For this purpose, the length or width of a rhombus 20 of braided structure G preferably corresponds to the distance between crossovers U of braiding material F located opposite each other in the axial direction or in the radial direction of braided structure G or of braid mandrel 4.

Control unit S reconciles a parameter captured by a testing means 18 with a pertinent specified or specifiable nominal parameter. One or a plurality of nominal parameters are, for example, made available or specified to control unit S by a data storage unit or a data base D for reconciliation. A negative reconciliation result exists if a captured parameter is not substantially identical to the pertinent nominal parameter or corresponds to it, or is not situated within an established tolerance range in comparison to the pertinent nominal parameter.

In case of a negative reconciliation result, in a fifth process step, the braiding operation is stopped by control unit S and device 1 is controlled in such a way that at least one section of the braided structure G is unbraided. This happens, for instance, by reverse relocation of one or a plurality of bobbins K and/or of braided structure G or braid mandrel 4. Advantageously, only that section of braided structure G is unbraided, which comprises the deviating parameter or corresponds to it.

In a sixth step, preferably by control unit S, a change of at least part of the configuration of commands, based on which the generation of the braided structure G was or is being carried out, is implemented.

By changing the configuration of commands, the deviating parameter is to be changed in such a way that a positive reconciliation result is achieved, i.e., that the captured parameter is substantially identical (or corresponding) or situated within an established tolerance range in comparison to the pertinent nominal parameter. In case of too short a rhombus length for example, the speed of the relocation of one or a plurality of bobbins K can be slowed down. Alternatively, and/or additionally, the speed of the relocation of braided structure G or a braid mandrel 4 can be increased.

The decision as to which parameter or parameters of the control of the braiding operation will be changed is preferably made automatically by control unit S or by an algorithm executed by it.

Particularly preferred, the change of the parameters or the change of the configuration of commands by an algorithm takes place based on or by taking into consideration certain provided information. Such information is, for instance, provided by data base D and/or comprises computer-readable commands for controlling a braiding machine for producing a braided structure. Alternatively, and/or additionally, the algorithm is arranged to use previously implemented and/or deposited parameter changes or changes of commands of the decision-making process as a basis.

Related to the selection of said at least one parameter, its capture, the reconciliation with at least one corresponding nominal parameter, with the unbraiding and/or with the change of the configuration of commands, algorithms based on artificial intelligence or machine learning can preferably be used.

In a next step, the braiding operation will be resumed and the braided structure will be generated according to the changed configuration of commands. Moreover, during the braiding operation, a capture of one or a plurality of parameters and an appropriate reconciliation with the pertinent nominal parameters takes place.

In case a positive reconciliation result exists, for example, if a captured parameter is substantially identical or corresponds or is situated within an established tolerance range in comparison to the pertinent nominal parameter. In this case, and if the braided structure has not yet been completed, the generation of braided structure G is resumed or continued.

If the braided structure is completed, in an additional step, a braiding program is created based on the last changed configuration of commands, i.e., according to the configuration of commands, in which no deviating parameter was captured. The braiding program is preferably conveyed to data base D to allow being taken into consideration in future decision-making processes of parameter changes.

The steps of a process according to one aspect of the disclosure, illustrated in FIG. 6 and described in the operation, correspond to an exemplary and particularly preferred embodiment and are primarily used for illustration purposes. In particular, a process according to one aspect of the disclosure may have additional and/or other steps and/or a changed sequence of steps.

1. Computer-aided process for creating a braiding program, the braiding program comprising machine-readable commands, which, during execution of the braiding program, induce a braiding machine to produce a braided structure (G), in particular, an at least partially braided implant, the process comprising:
a) generation of a braided structure (G) by braiding, in particular by braid-wrapping at least one braid mandrel (4) using a braiding material (F) according to a specified configuration of commands;
b) capturing at least one parameter of braided structure (G);
c) reconciling the captured parameter of braided structure (G) with at least one pertinent nominal parameter;
provided the captured parameter does not correspond to the pertinent nominal parameter:
c1-a) unbraiding at least one section of braided structure (G); and
c1-b) changing at least part of the configuration of commands for generating a braided structure (G) as well as repeating the steps a) to c) based on the changed configuration of commands;
provided the captured parameter corresponds to the pertinent nominal parameter;
c2) creating a braiding program according to the configuration of commands, in which the captured parameter corresponds to the pertinent nominal parameter.

2. Process according to claim 1, the change of at least part of the configuration of commands depending on:
type of the deviating parameter;
extent of the deviation of the parameter;
number of bobbins;
type of the braiding material (F);
thickness of the braiding material (F);
surface quality of the braiding material (F);
surface quality of a braid mandrel (4); and/or
geometry of a braid mandrel (4).

3. Process according to claim 1, the change of the configuration of commands being influenced by a teachable computer-implemented algorithm.

4. Process according to claim 3, the teachable, computer-implemented algorithm changing the configuration of commands based on:
a specified configuration of commands;
a previously captured parameter deviation;
an implemented configuration change; and/or
a detected effect of a specific configuration change.

5. Process according to claim 1, the configuration of commands for generating the braided structure comprising one or a plurality of the following parameters:
relocation of at least one bobbin (K);
relocation of the braided structure (G);
relocation of a braid mandrel (4);
position of a braid ring (8);
position of a braid eye (10);
tension of the braiding material (F).

6. Process according to claim 1, a parameter of the braided structure (G) comprising:
position of at least one crossover of braiding material (F), in particular in relation to at least one feature of a braid mandrel (4);
diameter of the braiding material (F);
length of a rhombus (20);
width of a rhombus (20);
length of a rhombus segment (22); and/or
braid angle of a rhombus (20).

7. Process according to claim 1, the capture of a parameter of the braided structure (G) taking place in at least one specific section of the braided structure (G), preferably in relation to a braid mandrel (4).

8. Process according to claim 1, the unbraiding comprising a substantially reversed relocation of at least one of: at least one bobbin (K), the braided structure (G) and a braid mandrel (4).

9. Process according to claim 1, the unbraiding taking place at least up to the section of the braided structure (G), which has the parameter that deviates from the pertinent nominal parameter.

10. A computer program comprising commands which, during execution of the program by a device, induce the latter to execute the process for creating a braiding program according to claim 1.

11. Computer-implemented braiding program comprising commands, which, during execution of the braiding program, induce a braiding machine (1) to produce a braided structure (G), in particular an at least partially braided implant, the braiding program having been created by the process according to claim 1.

12. Device (1) for creating a braiding program, the braiding program comprising machine-readable commands, which, during execution of the braiding program, induce a braiding machine (1) to produce a braided structure (G), in particular an at least partially braided implant, the device (1) being particularly suitable for implementing the process according to claim 1 and comprising:
at least one bobbin (K) for guiding a braiding material (F), said at least one bobbin (K) being relocatable to move along paths, in particular of randomly specifiable paths;
a relocation device (2) for relocating said at least one bobbin (K) along a path;
a puller (6) for relocating braided structure (G), in particular for picking up and relocating at least one braid mandrel (4);
a control unit for controlling the relocation of said at least one bobbin (K) and/or relocation of braided structure (G), in particular of said at least one braid mandrel (4), in order to generate in this manner a braided structure (G) by braiding, in particular, by braid-wrapping at least one braid mandrel (4) using the braiding material (F); and
a testing means (18) for capturing at least one parameter of a braided structure (G) generated by device (1);
the control unit being furthermore arranged for:
a) generating a braided structure (G) by braiding, in particular by braid-wrapping at least one braid mandrel (4) using a braiding material (F) according to a specified configuration of commands;
b) capturing at least one parameter of braided structure (G);
c) reconciling said at least one captured parameter of braided structure (G) with at least one pertinent nominal parameter;
provided the captured parameter does not correspond to the pertinent nominal parameter:
c1-a) unbraiding at least one section of braided structure (G); and
c1-b) changing at least part of the configuration of commands for generating a braided structure (G) as well as repeating the steps a) to c) based on the changed configuration of commands;
provided the captured parameter corresponds to the pertinent nominal parameter:
c2) creating a braiding program according to the configuration of commands, in which the captured parameter corresponds to the pertinent nominal parameter.

13. Process according to claim 7, the capture of a parameter of the braided structure (G) taking place in particular in an aerial section (12), in a dropping section (14) and/or in a fastening section (16) of the braided structure.
